(19) United States
(12) Patent Application Publication
(10) Pub. No.: US 2009/0280074 A1
(43) Pub. Date: Nov. 12, 2009

(54) USE OF GLYCYRRHETIC ACID AND/OR GLYCYRRHIZIN FOR PRODUCING COSMETIC PREPARATIONS FOR TANNING THE SKIN

(76) Inventors: Svenja Gschwind, Therwil (CH); Wolfram Gerwat, Hamburg (DE); Cathrin Scherner, Norderstedt (DE); Rainer Wolber, Hamburg (DE)

Correspondence Address: GREENBLUM & BERNSTEIN, P.L.C., 1950 ROLAND CLARKE PLACE, RESTON, VA, 20191, US

(21) Appl. No.: 12/281,116

(22) PCT Filed: Feb. 23, 2007
(86) PCT No.: PCT/EP07/01561

§ 371 Date: Dec. 28, 2008

(30) Foreign Application Priority Data
Mar. 1, 2006 (DE) 10 2006 009 850.1

(51) Int. Cl. A61K 8/18; A61Q 17/04

(52) U.S. Cl. 424/59

(57) ABSTRACT

The use of glycyrrhetin and/or glycyrrhizin for producing cosmetic or dermatological formulations for enhancing natural skin browning.

The present invention relates to cosmetic and dermatological preparations for tanning the skin, in particular preparations that offer protection against UV radiation at the same time.

The damaging action of the ultraviolet part of solar radiation on the skin is generally known. Whereas rays with a wavelength which is smaller than 290 nm (the so-called UVC range) are absorbed by the ozone layer in the earth's atmosphere, rays in the range between 290 nm and 320 nm, the so-called UVB range, cause erythema, simple sunburn or even more or less severe burns.

The narrower range around 308 nm is indicated as a maximum of the erythema activity of sunlight.

Numerous compounds are known for the protection against UVB radiation, which compounds are mostly derivatives of 3-benzylidenecamphor, 4-aminobenzoic acid, cinnamic acid, salicylic acid, benzophenone as well as of 2-phenylbenzimidazole.

It is important also to have filter substances available for the range between about 320 nm and about 400 nm, the so-called UVA range, since rays of this range can also cause damage. It has thus been proved that UVA radiation leads to a damaging of the elastic and collagen fibers of the connective tissue, which causes the skin to age prematurely, and that UVA radiation is to be considered the cause of numerous phototoxic and photoallergic reactions. The damaging effect of UVB radiation can be intensified by UVA radiation.

UVA radiation can also cause skin damage by damaging the keratin or elastin present in the skin. As a result, the elasticity and the ability of the skin to store water are reduced, i.e. the skin becomes less supple and tends towards wrinkling. The strikingly high incidence of skin cancer in regions where solar radiation is strong indicates that damage to the genetic information in the cells is also obviously caused by sunlight, in particular by UVA radiation.

However, UV radiation can also lead to photochemical reactions, in which case the photochemical reaction products intervene in the skin's metabolism.

Such photochemical reaction products are predominantly free-radical compounds, e.g., hydroxyl radicals. Undefined free-radical photo-products which are formed in the skin itself can also display uncontrolled secondary reactions because of their high reactivity. However, singlet oxygen, a non-radical excited state of the oxygen molecule, can also be formed during UV irradiation, as can short-lived epoxides and many others. Singlet oxygen, for example, differs from the normally present triplet oxygen (free-radical ground state) by its increased reactivity. However, excited, reactive (free-radical) triplet states of the oxygen molecule also exist.

UV radiation is also a type of ionizing radiation. There is therefore the risk that UV exposure may also produce ionic species, which then, for their part, are capable of oxidative intervention in the biochemical processes.

The pigmentation of human skin is essentially brought about by the presence of melanin. Melanin and its degradation products (melanoids), carotene, the degree of perfusion, and the condition and thickness of the stratum corneum and other skin layers permit skin shades from virtually white (in cases of reduced filling or in cases of an absence of blood vessels) or yellowish via pale brown-reddish, bluish to brown of different shades and finally almost black. The individual regions of the skin display differing depths of shade as a result of varying amounts of melanin.

Natural melanin protects the skin from penetrating UV radiation. The number of melanin granules produced in the melanocytes determines whether a person has pale skin or dark skin. In cases of strong pigmentation (e.g., in colored races, but also in those with pale skin following some UV irradiation), melanin is also to be found in the stratum spinosum and even in the stratum corneum. It attenuates the UV radiation by up to about 90% before it reaches the corium.

Depending on their sensitivity to light, the following skin types are generally distinguished:

Skin type I never tans, always burns.
Skin type II hardly tans, burns easily.
Skin type III tans averagely well.
Skin type IV tans easily and lastingly, almost never burns.
Skin type V dark, often almost black skin, never burns.

The natural shielding against harmful UV radiation is a manifest advantage of natural skin tanning. For a number of decades now, moreover, a “healthy” skin color has been seen as a sign of athletic activity, in particular, and is therefore regarded as desirable by a broad stratum of consumers. Representatives of skin types I and II who wish to enjoy this type of skin coloring are therefore driven in any case to rely on self-tanning products. However, representatives of skin type III who do not want to be exposed excessively to the risks of sunbathing but nevertheless want to appear tanned, are also appreciative target groups for self-tanning preparations.

Artificial skin tanning can be brought about by cosmetic or medicinal means, with the following approaches essentially playing a part:

The regular intake of carotene products results in carotene being stored in the subcutaneous fatty tissue, and the skin gradually turns orange to yellow-brown.

With the aid of make-up products which can be removed by washing it is possible to achieve a slight skin coloring (e.g., extracts of fresh green walnut shells, henna).

Coloring can also be accomplished by means of a chemical change in the horny layer of the skin using so-called self-tanning preparations. The principal active substance is dihydroxyacetone (DHA). The tan achieved in this way cannot be removed by washing and comes off only with the normal flaking of the skin (after about 10-15 days). Dihydroxyacetone can be referred to as ketotriose and, as a reducing sugar, reacts with the amino acids of the skin and with the free amino and imino groups of keratin, respectively, via a series of intermediate stages, in a Maillard reaction, to form brown-colored substances, referred to as melanoids, which are occasionally also called melanoidins.

A disadvantage of tanning with dihydroxyacetone is that, unlike “sun-tanned” skin, skin tanned with DHA is not protected from sunburn.

The use of glycyrrhetic acid and glycyrrhizin in cosmetics and dermatics is known per se. Glycyrrhetic acid is characterized by the following structure:

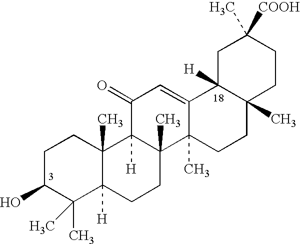

In addition to the naturally occurring 18β form there is also an 18α form. The glycyrrhetic acid that can be isolated from licorice (glycyrrhiza glabra, leguminosae) is the aglycon of glycyrrhizin (which represents the 2β-glucuronido-α-glucuronid of glycyrrhetic acid).

Glycyrrhizin is characterized by the following structure:

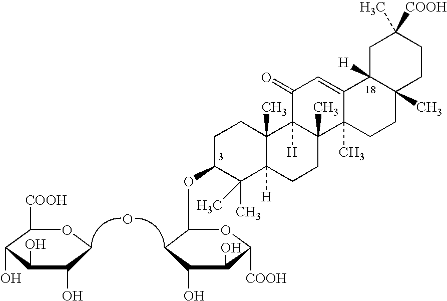

Glycyrrhetic acid is used as an anti-inflammatory agent. Glycyrrhetic acid-3-hemisuccinate is used as a therapeutic agent for ulcers.

Glycyrrhizin has an anti-inflammatory effect and serves as a cough remedy and expectorant in the form of licorice juice (sirupus liquiritiae).

Glycyrrhetic acid and glycyrrhizin are known to one skilled in the art as an agent with a skin-lightening effect.

The object of the present invention was therefore to remedy the disadvantages of the prior art.

It was, however, surprising and not predictable for one skilled in the art that the use of glycyrrhetic acid, glycyrrhizin and/or the corresponding sodium salts, ammonium salts and potassium salts and/or the corresponding esters for producing cosmetic preparations

It had not been predictable for one skilled in the art that the preparations according to the invention would increase the pigmentation of the skin, render possible a more uniform tanning of the skin, would not develop any undesired smells during application to the skin and would avoid a discoloration of clothes after application to the skin.

Advantageously, skin care products according to the invention contain 0.0001%-20% by weight of glycyrrhetin and/or glycyrrhizin.

Preferably, cosmetic or dermatological preparations according to the invention contain 0.001%-10% by weight of glycyrrhetin and/or glycyrrhizin, based on the overall composition of the preparations.

Particularly preferably, cosmetic or dermatological preparations according to the invention contain 0.01%-1% by weight of glycyrrhetin and/or glycyrrhizin, based on the overall composition of the preparations.

Emulsions according to the invention for the purposes of the present invention, e.g., in the form of a cream, a lotion, a cosmetic milk are advantageous and contain, e.g., fats, oils, waxes and/or other fatty substances, as well as water and one or more emulsifiers, of the kind customarily used for a formulation of this type.

It is also possible and advantageous for the purposes of the present invention to introduce the active substance used according to the invention into aqueous systems or surfactant preparations for the cleansing of skin and hair.

One skilled in the art is, of course, aware that high-quality cosmetic compositions are in most cases inconceivable without the use of the usual auxiliaries and additives. These include, for example, builders, fillers, perfume, dyes, emulsifiers, additional active substances such as vitamins or proteins, light protection agents, stabilizers, insect repellents, alcohol, water, salts, substances having anti-microbial, proteolytic or keratolytic activity, etc.

Corresponding requirements apply mutatis mutandis for the formulation of medicinal preparations.

Medicinal topical compositions for the purposes of the present invention generally comprise one or more medicaments in an effective concentration. For the sake of simplicity, for a clear distinction between cosmetic and medicinal application and corresponding products, reference is made to the legal provisions of the Federal Republic of Germany (e.g., Cosmetics Directive, Foods and Drugs Act).

It is thereby likewise advantageous to add the active substance used according to the invention as additive to preparations that already contain other active substances for other purposes.

The cosmetic and/or dermatological formulations according to the invention can be composed as usual and be used for the treatment of the skin and/or the hair for the purposes of a dermatological treatment or a treatment for the purposes of skin care cosmetics. However, they may also be used in make-up products in decorative cosmetics or in cosmetic and dermatological cleansing products.

If the cosmetic or dermatological preparation is a solution or lotion, the following solvents can be used: water or aqueous solutions, furthermore oils, such as triglycerides of capric or caprylic acid, but preferably castor oil, fats, waxes and other natural and synthetic fatty substances, preferably esters of fatty acids with alcohols of low C number, e.g., with isopropanol, propylene glycol or glycerol, or esters of fatty alcohols with alkanoic acids of low carbon number or with fatty acids, but also alcohols, dioles or polyols of low C number, as well as ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular mixtures of the solvents mentioned above are used, just as water can be an advantageous further component with alcoholic solvents.

Cosmetic and dermatological preparations according to the invention, also, e.g., for the protection of the skin against UV radiation, can be present in different forms, such as those, e.g., customarily used for this type of preparations. They may thus represent, e.g., a solution, an emulsion of the water-in-oil (W/O) type or of the oil-in-water (O/W) type, or a multiple emulsion, for example of the type water-in-oil-in-water (W/O/W), oil-in-water-in-oil (O/W/O), a gel, a hydrodispersion, a lamellar phase, a liquid isotropic solution phase, a micellar phase, a solid or dispersed single or multiple hexagonal phase, a solid or dispersed single or multiple cubic phase, a lyotropic phase, a crystalline phase, a solid stick or also an aerosol.

The active substances according to the invention can also be used particularly advantageously in microemulsions, for example as described in the German published patent application DE-195 9 079.

The oil phase of the preparations according to the invention is advantageously chosen from the group of esters of saturated and/or unsaturated, branched and/or unbranched alkanecarboxylic acids with a chain length of from 3 to 30 C atoms and saturated and/or unsaturated, branched and/or unbranched alcohols with a chain length of from 3 to 30 C atoms, from the group of esters of aromatic carboxylic acids and saturated and/or unsaturated, branched and/or unbranched alcohols with a chain length of from 3 to 30 C atoms. Such ester oils can then be advantageously chosen from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g., jojoba oil.

In addition, the oil phase can advantageously be chosen from the group of branched and unbranched hydrocarbons and hydrocarbon waxes, of silicone oils, of dialkyl ethers, the group of saturated or unsaturated, branched or unbranched alcohols, and of fatty acid triglycerides, namely the triglycerol esters of saturated and/or unsaturated, branched and/or unbranched alkanecarboxylic acids having a chain length of from 8 to 24, in particular 12-18, C atoms. The fatty acid triglycerides can, for example, advantageously be chosen from the group of synthetic, semisynthetic and natural oils, e.g. olive oil, sunflower oil, soybean oil, peanut oil, rapeseed oil, almond oil, palm oil, coconut oil, palm kernel oil and the like.

Any blends of oil and wax components of this type can also be used advantageously for the purposes of the present invention.

The oil phase is advantageously chosen from the group 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride, dicaprylyl ether.

Mixtures of Cl₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, for the purposes of the present invention, paraffin oil, squalane and squalene may be used advantageously.

The oil phase can also advantageously have a content of cyclic or linear silicone oils, or be composed entirely of oils of this type, although it is preferred to use an additional content of other oil phase components apart from the silicone oil or the silicone oils.

Cyclomethicone (octamethylcyclotetrasiloxane) is advantageously used as silicone oil to be used according to the invention. However, other silicone oils are also to be used advantageously for the purposes of the present invention, for example, hexamethylcyclotrisiloxane, polydimethylsiloxane, poly(methylphenylsiloxane).

Also particularly advantageous are mixtures of cyclomethicone and isotridecyl isononanoate, of cyclomethicone and 2-ethylhexyl isostearate.

Advantageously, the content of the oil phase is between 1% and 50% by weight, based on the total weight of the preparations, preferably 2.5%-30% by weight, particularly preferably 5%-15% by weight.

In terms of application, the cosmetic and/or dermatological formulations according to the invention are applied to the skin and/or the hair in a sufficient amount in a manner customary for cosmetics and dermatics.

Accordingly, preparations for the purposes of the present invention preferably contain at least one further UVA, UVB and/or broadband filter substance. Although this is not obligatory, the formulations can contain, if necessary, also one or more organic and/or inorganic pigments as UV filter substances which can be present in the aqueous phase and/or the oil phase.

The preparations according to the invention can further advantageously also be present in the form of so-called oil-free cosmetic or dermatological emulsions which contain an aqueous phase and at least one UV filter substance liquid at room temperature as a further phase.

Particularly advantageous UV filter substances which are liquid at room temperature for the purposes of the present invention are homomethyl salicylate (INCI: Homosalate), 2-ethylhexyl 2-cyano-3,3-diphenylacrylate (INCI: octocrylene), 2-ethylhexyl-2-hydroxybenzoate (2-ethylhexyl salicylate, octyl salicylate, INCI: octyl salicylate) and esters of cinnamic acid, preferably 2-ethylhexyl 4-methoxycinnamate (INCI: octyl methoxycinnamate) and isopentyl 4-methoxycinnamate (INCI: isoamyl p-methoxycinnamate).

Advantageous UVA filter substances for the purposes of the present invention are dibenzoylmethane derivatives, in particular 4-(tert-butyl)-4′-methoxydibenzoylmethane (CAS No. 70356-09-1), which is sold by Givaudan under the trade name Parsol® 1789 and by Merck under the trade name Eusolex® 9020.

Further advantageous UV filter substances for the purposes of the present invention are sulfonated, water-soluble UV filters, such as, e.g.:
phenylene-1,4-bis(2-benzimidazyl)-3,3′-5,5′-tetrasulfonic acid and salts thereof, particularly the corresponding sodium salts, potassium salts or triethanolammonium salts, in particular the phenylene-1,4-bis(2-benzimidazyl)-3,3′-5,5′-tetrasulfonic acid bis-sodium salt having the INCI name Bisimidazylate (CAS No. 180898-37-7), which is available, for example, under the trade name Neo Heliopan AP from Haarmann & Reimer;
salts of 2-phenylbenzimidazole-5-sulfonic acid, such as its sodium salt, potassium salt or its triethanolammonium salt, and the sulfonic acid itself with the INCI name phenylbenzimidazole sulfonic acid (CAS No. 27503-81-7), which is available, for example, under the trade name Eusolex 232 from Merck or under the trade name Neo Heliopan Hydro from Haarmann & Reimer;
1,4-di(2-oxo-10-sulfo-3-bornylidenemethyl)-benzene (also: 3,3′-(1,4-phenylenedimethylene)-bis-(7,7-dimethyl-2-oxo-bicyclo-[2.2.1]hept-1-ylmethane sulfonic acid) and salts thereof (in particular the corresponding 10-sulfato compounds, in particular the corresponding sodium salt, potassium salt or triethanolammonium salt), which is also known as benzene-1,4-di(2-oxo-3-bornylidenemethyl-10-sulfonic acid). Benzene-1,4-di(2-oxo-3-bornylidenemethyl-10-sulfonic acid) has the INCI name terephthalidene dicamphor sulfonic acid (CAS No.: 90457-82-2) and is available, for example, under the trade name Mexoryl SX from Chimex;
sulfonic acid derivatives of 3-benzylidene camphor, such as, e.g., 4-(2-oxo-3-bornylidenemethyl)benzenesulfonic acid, 2-methyl-5-(2-oxo-3-bornylidenemethyl)sulfonic acid and salts thereof.

Advantageous UV filter substances for the purposes of the present invention include furthermore so-called broadband filters, i.e., filter substances which absorb both UVA and UVB radiation.

Advantageous broadband filters or UVB filter substances are, for example, triazine derivatives, such as, e.g.,
diethylhexylbutylamidotriazone (INCI: diethylhexylbutamidotriazone), which is available under the trade name UVASORB HEB from Sigma 3V;
tris(2-ethylhexyl) 4,4′,4″-(1,3,5-triazine-2,4,6-triyltriimino)-tris-benzoate, also: 2,4,6-tris-[anilino-(p-carbo-2′-ethyl-1′-hexyloxy)]-1,3,5-triazine (INCI:
ethylhexyl triazone), which is sold by BASF Aktiengesellschaft under the trade name UVINUL® T 150.

Another advantageous broadband filter for the purposes of the present invention is 2,2′-methylene-bis-(6-(2H-benzotriazol-2-yl)-4-(1,1,3,3-tetramethylbutyl)-phenol) which is available under the trade name Tinosorb® M from CIBA-Chemikalien GmbH.

Another advantageous broadband filter for the purposes of the present invention is 2-(2H-benzotriazol-2-yl)-4-methyl-6-[2-methyl-3-[1,3,3,3-tetramethyl-1-[(trimethylsilyl)oxy]-disiloxanyl]propyl]-phenol (CAS No.: 155633-54-8) with the INCI name drometrizole trisiloxane, which is available under the trade name Mexoryl® XL from Chimex.

The further UV filter substances may be oil-soluble or water-soluble.

Advantageous oil-soluble UVB and/or broadband filter substances for the purposes of the present invention are, e.g.:
3-benzylidene camphor derivatives, preferably 3-(4-methylbenzylidene) camphor, 3-benzylidene camphor;
cinnamic acid derivatives, preferably 2-ethylhexylmethoxycinnamate (CAS No.: 5466-77-3), which is available under the trade name Parsol MCX from Givaudan.
4-aminobenzoic acid derivatives, preferably (2-ethylhexyl) 4-(dimethylamino)benzoate, amyl 4-(dimethylamino)benzoate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone
and UV filters bound to polymers.
3-(4-(2,2-bisethoxycarbonylvinyl)-phenoxy)propenyl)-methoxysiloxane/dimethylsiloxane copolymer, which is available, for example, under the trade name Parsol® SLX from Hoffmann La Roche.

Advantageous water-soluble filter substances are, e.g.:

sulfonic acid derivatives of 3-benzylidene camphor, such as, e.g., 4-(2-oxo-3-bornylidenemethyl)benzenesulfonic acid, 2-methyl-5-(2-oxo-3-bornylidenemethyl)sulfonic acid and salts thereof.

A further light protection filter substance which may advantageously be used according to the invention is ethylhexyl-2-cyano-3,3-diphenylacrylate (octocrylene), which is available from BASF under the name Uvinul® N 539. Particularly advantageous preparations for the purposes of the present invention which are characterized by a high or very high UVA and/or UVB protection furthermore preferably contain, in addition to the filter substance(s) according to the invention, further UVA and/or broadband filters, in particular dibenzoylmethane derivatives [for example, 4-(tert-butyl)-4′-methoxydibenzoylmethane], phenylene-1,4-bis-(2-benzimidazyl)-3,3′-5,5′-tetrasulfonic acid and/or salts thereof, 1,4-di(2-oxo-10-sulfo-3-bornylidenemethyl)-benzene and/or salts thereof, in each case individually or in any desired combinations with one another.

The above list of UV filters which can be employed for the purposes of the present invention is of course not intended to be limiting.

The preparations according to the invention advantageously comprise the substances which absorb UV radiation in the UVA and/or UVB range in a total amount of, e.g., from 0.1% by weight to 30% by weight, preferably from 0.5% to 20% by weight, in particular from 1.0% to 15.0% by weight, in each case based on the total weight of the preparations, in order to provide cosmetic preparations which protect the hair or the skin from the entire range of ultraviolet radiation. Further active substances that are to be used advantageously for the purposes of the present invention are those that have a positive effect on the condition of the skin, such as in particular active substances for positively influencing aging skin which reduce the development of lines or even existing lines. Advantageous are in particular bioquinones, in particular ubiquinone (Q10), folic acid and its derivatives (in particular tetrahydrofolic acid and dihydrofolic acid), niacin and its derivatives (in particular niacinamide), creatine and creatinine, camitine, biotin, isoflavone, hop and hop-malt extracts. Agents which promote the restructuring of connective tissue, such as natural and synthetic isoflavonoids and isoflavonoid-containing plant extracts—such as, e.g., soya and clover extracts—can also be used very well in the formulations according to the invention. It is also found that the formulations are particularly suitable for using active substances for aiding the skin functions in dry skin (such as, for example, vitamin C, biotin, camitine, green tea extracts, eucalyptus oil, urea and mineral salts (such as, e.g., NaCl, sea minerals) and osmolytes (such as, e.g., inositol, betaine, quaternary ammonium compounds). In a similar way, the incorporation of active substances for alleviating or positively influencing irritative skin conditions, whether for sensitive skin in general or for skin irritated by noxae (UV light, chemicals) has proven to be advantageous. Mention is made here of active substances such as sericosides, various extracts of licorice, licochalcones, in particular licochalcone A, silymarin, silyphos, dexpanthenol, inhibitors of the prostaglandin metabolism, in particular of cyclooxygenase and of the leukotriene metabolism, in particular of 5-lipoxygenase, but also of the 5-lipoxygenase inhibitor protein, FLAP. Furthermore, the active substances mentioned above are particularly suitable for creating or renewing an (energy) depot and activating the repair of different cellular structures, in particular the DNA.

Particularly advantageous preparations are further obtained if antioxidants are used as additives or active substances. According to the invention, the preparations advantageously contain one or more antioxidants. Any antioxidants which are suitable or conventional for cosmetic and dermatological applications can be used as favorable antioxidants, but are to be used in an optional manner.

Of particular advantage for the purposes of the present invention is the use of water-soluble antioxidants such as, for example, vitamins, e.g., ascorbic acid and derivatives thereof—in particular ascorbyl palmitate, Na and Mg ascorbyl phosphate and ascorbyl acetate—as well as rutinic acid and derivatives thereof, in particular alpha-glucosyl rutin, quercetin and isoquercetin.

Particularly preferred antioxidants are further vitamin E and derivatives thereof (in particular vitamin E acetate), vitamin A and derivatives thereof (in particular vitamin A palmitate) and carnosine, butylated hydroxytoluene, butylated hydroxyanisole, beta-alanine and carotenoides (in particular beta-carotene) and phytoene.

The amount of antioxidants (one or more compounds) in the preparations is preferably 0.001% to 30% by weight, particularly preferably 0.05% to 20% by weight, in particular 0.1% to 10% by weight, based on the total weight of the preparation.

It is particularly advantageous if the cosmetic preparations according to the present invention contain cosmetic or dermatological active substances, with the preferred active substances being antioxidants that can protect the skin against oxidative stress.

The preparations according to the present invention can further advantageously contain self-tanning substances such as, for example, dihydroxyacetone, melanin derivatives and erythrulose, with these substances being used in concentrations of from 0.1% by weight to 8% by weight, preferably from 0.4%-2% by weight, based on the total weight of the preparation. In addition to dihydroxyacetone, the formulations according to the invention can further advantageously contain also nut extracts and other substances that are to maintain the tan, produce it or additionally intensify it. The combination of self-tanners, in particular dihydroxyacetone and erythrulose, with the compounds according to the invention in cosmetic preparations has proved to be extremely advantageous for treating undesired hyperpigmentation (melasma, age spots, freckles, etc.).

The following examples are to illustrate the embodiments of the present inventions.

Lotion

| Water | ad 100 |
|---|---|
| Stearic acid | 2 |
| Dicaprylyl ether | 3 |
| Octyldodecanol | 2 |
| C12-15 Alkyl benzoate | 4 |
| Cetyl alcohol | 2 |
| Cetearyl ethylhexanoate + isopropyl myristate | 2 |
| Glycerol | 5 |
| Ethylhexyl methoxycinnamate | 2 |
| TiO2 | 1 |
| Cetyl palmitate | 1 |
| Glyceryl stearate | 1 |
| Phenoxyethanol | 0.4 |
| Butyl methoxydibenzoylmethane | 2 |
| Perfume | q.s. |
| EDTA | 0.2 |
| Carbomer | 0.2 |
| Magnesium aluminum silicar | 0.2 |
| Methyl paraben | 0.1 |
| Vitamin E acetate | 0.1 |
| Glycyrrhetic acid | 0.1 |
| Propyl paraben | 0.05 |
| BHT | 0.05 |
| DHA | 1 |

Night cream

| | % by weight |
|---|---|
| Glyceryl stearate citrate | 2 |
| Cetyl ricinoleate | 1 |
| Stearyl alcohol | 2 |
| Cetyl alcohol | 2 |
| Hydrogenated coco glycerides | 1 |
| Caprylic acid/capric acid triglycerides | 3 |
| Ethylhexyl cocoate | 2 |
| Dicaprylyl ether | 2 |
| C12-15 Alkyl benzoate | 3 |
| Tocopheryl acetate | 1 |
| Ubiquinone (coenzyme Q10) | 0.05 |
| Sodium ascorbyl phosphate | 0.1 |
| Glycyrrhetic acid | 0.1 |
| p-Hydroxybenzoic acid alkyl ester (paraben) | 0.4 |
| Methyl propanediol | 1 |
| Carrageenan | 0.1 |
| Carbomer | 0.2 |
| EDTA | 0.2 |
| Glycerol | 7 |
| Water-soluble and/or oil-soluble colorants | 0.05 |
| Fillers/additives (nylon, BHT) | 0.5 |
| Perfume | q.s. |
| Water | ad 100 |

Day cream

| | % by weight |
|---|---|
| PEG-40 stearate | 1 |
| Glyceryl stearate | 3 |
| Cetearyl alcohol | 2 |
| Dimeticone | 1 |
| Hydrogenated coco glycerides | 2 |
| Caprylic acid/capric acid triglycerides | 2 |
| Octyldodecanol | 2 |
| Dicaprylyl carbonate | 2 |
| C12-15 Alkyl benzoate | 3 |
| Ethylhexyl methoxycinnamate | 4 |
| Butyl methoxydibenzoylmethane | 2 |
| Tocopheryl acetate | 1 |
| Panthenol | 0.5 |
| Sodium ascorbyl phosphate | 0.1 |
| Glycyrrhetic acid | 0.1 |
| p-Hydroxybenzoic acid alkyl ester (paraben) | 0.4 |
| Methyl propanediol | 1 |
| Carbomer | 0.2 |
| Xanthan gum | 0.1 |
| EDTA | 0.2 |
| Glycerol | 8 |
| Tapioca starch | 0.05 |
| Fillers/additives | 0.3 |
| Perfume | q.s. |
| Water | ad 100 |

Face cream

| | |
|---|---|
| Polyglyceryl-3-methylglucose distearate | 2 |
| Sorbitan stearate | 3 |
| Cetyl alcohol | 2 |
| Myristyl myristate | 1 |
| Dicaprylyl ether | 3 |
| Octyldodecanol | 2 |
| C12-15 Alkyl benzoate | 3 |
| Cetearyl ethylhexanoate + isopropyl myristate | 2 |
| Ethylhexyl methoxycinnamate | 2 |
| Ethylhexyl triazone | 1 |
| Butyl methoxydibenzoylmethane | 2 |
| Magnesium aluminum silicar | 0.2 |
| Glycerol | 5 |
| Phenoxyethanol | 0.4 |
| Parabens | 0.3 |
| Vitamin E acetate | 0.1 |
| Glycyrrhetic acid | 0.1 |
| BHT | 0.05 |
| EDTA | 0.2 |
| Carbomer | 0.2 |
| Perfume | q.s. |
| Water | ad 100 |

1.-4. (canceled)

5. A method of enhancing natural tanning of skin, wherein the method comprises applying to the skin a cosmetic or dermatological preparation which comprises at least one of glycyrrhetic acid and glycyrrhizin in an amount which is effective for enhancing natural skin tanning.

6. The method of claim 5, wherein the preparation comprises at least 0.0001% by weight of at least one of glycyrrhetic acid and glycyrrhizin, based on a total weight of the preparation.

7. The method of claim 6, wherein the preparation comprises not more than 20% by weight of at least one of glycyrrhetic acid and glycyrrhizin.

8. The method of claim 5, wherein the preparation comprises at least 0.001% by weight of at least one of glycyrrhetic acid and glycyrrhizin.

9. The method of claim 8, wherein the preparation comprises not more than 10% by weight of at least one of glycyrrhetic acid and glycyrrhizin.

10. The method of claim 5, wherein the preparation comprises at least 0.01% by weight of at least one of glycyrrhetic acid and glycyrrhizin.

11. The method of claim 10, wherein the preparation comprises not more than 1% by weight of at least one of glycyrrhetic acid and glycyrrhizin.

12. The method of claim 7, wherein the preparation comprises at least glycyrrhetic acid.

13. The method of claim 7, wherein the preparation comprises at least glycyrrhizin.

14. The method of claim 4, wherein the preparation further comprises at least one UV filter substance.

15. The method of claim 14, wherein the at least one UV filter substance comprises at least one of ethylhexyl methoxycinnamate, butyl methoxydibenzoylmethane, and ethylhexyl triazone.

16. The method of claim 5, wherein the preparation further comprises at least one self-tanning substance.

17. The method of claim 16, wherein the at least one self-tanning substance comprises at least one of dihydroxyacetone and erythrulose.

18. The method of claim 17, wherein the at least one self-tanning substance is present in an amount of from 0.1% to 8% by weight, based on a total weight of the preparation.

19. The method of claim 5, wherein the preparation further comprises at least one antioxidant.

20. The method of claim 19, wherein the at least one antioxidant comprises at least one of ascorbic acid, ascorbyl palmitate, Na or Mg ascorbyl phosphate, ascorbyl acetate, rutinic acid, alpha-glucosyl rutin, quercetin, isoquercetin, vitamin E and derivatives thereof, vitamin A and derivatives thereof, carnosine, butylated hydroxytoluene, butylated hydroxyanisole, beta-alanine, a carotenoide and phytoene.

21. A method of treating hyperpigmentation of skin, wherein the method comprises applying to the skin a cosmetic or dermatological preparation which comprises at least one of glycyrrhetic acid and glycyrrhizin and at least one self-tanning substance.

22. A cosmetic or dermatological preparation which comprises at least one of glycyrrhetic acid and glycyrrhizin in an amount of from 0.0001% to 20% by weight, based on a total weight of the preparation, and further comprises at least one substance which is selected from UV filter substances, self-tanning agents and antioxidants.

23. The preparation of claim 22, wherein the preparation comprises at least glycyrrhetic acid.

24. The preparation of claim 23, wherein the preparation comprises at least one UV filter substance.
